(19) United States
(12) Patent Application Publication
(10) Pub. No.: US 2022/0211491 A1
(43) Pub. Date: Jul. 7, 2022

(54) AN ADDITIONAL IMPROVEMENT IN MANAGEMENT OF THROMBOEMBOLISM RISK THAT MAY BE CAUSED BY A MEDICAL DEVICE BEING INSERTED INTRAVASCULARLY AND PROVIDING PROTECTION AGAINST PULMONARY HYPERTENSION RISK IN TREATMENT OF PATIENTS WITH HEART DEFECT

(71) Applicant: Mehmet GUMUSTAS, ANKARA (TR)

(72) Inventor: Mehmet GUMUSTAS, ANKARA (TR)

(21) Appl. No.: 17/611,409

(22) PCT Filed: Jul. 29, 2019
(86) PCT No.: PCT/TR2019/050632

§ 371 Date: Nov. 15, 2021

(30) Foreign Application Priority Data
May 20, 2019 (TR) 2019/07586

(51) Int. Cl. A61F 2/24; A61B 5/0215

(52) U.S. Cl. CPC A61F 2/2418; A61F 2/2475; A61F 2250/0039; A61F 2230/0067; A61B 5/0215

(57) ABSTRACT

The present invention relates to at least three orifices which are situated at the junction level of the proximal end close to the heart and the distal end of the medical device and on the cuffed distal end, in order to ensure control of a potential thromboembolism risk, after implantation of the medical device which is used for the purpose of palliative treatment and/or treatment without developing pulmonary hypertension in patients of all age groups who have human heart defects, particularly congenital heart defects, secondary increased pulmonary blood flow and pulmonary hypertension risk; and preparing the left ventricular for systemic circulation in case of development of right ventricular failure following atrial “switch” in patients who have transposition of large arteries with a left ventricular being subjected to involution—to the main pulmonary artery.

### TECHNICAL FIELD

The present invention relates to an additional improvement and solution in management of potential thromboembolism risk that may he caused by the medical device which is used for the purpose of palliative treatment and/or treatment without developing pulmonary hypertension in patients of all age groups who have human heart defects, particularly congenital heart defects, secondary increased pulmonary blood flow and pulmonary hypertension risk; and preparing the left ventricular for systemic circulation in case of development of right ventricular failure following atrial “switch” in patients who have transposition of large arteries with a left ventricular being subjected to involution.

### BACKGROUND OF THE INVENTION

Today, main pulmonary artery is intervened by materials that provide pressure extravascularly by means of open heart surgery in patients with congenital heart defects having pulmonary hypertension risk and this approach is called as “pulmonary artery banding operation” in literature. Although post-operative pulmonary pressure is controlled, a second surgical intervention is absolutely required for every patient in this classical method. Cost, applicability and complication risk of this process are quite high.

The “pulmonary artery banding operation”, which was first applied in 1951, takes a very important place in palliation of secondary increased pulmonary blood flow for congenital heart diseases today. Being a conventional approach, the “pulmonary artery banding operation” is a surgical treatment and. its results are controversial particularly in newborns and infants. Although pulmonary artery banding operation is simple in terms of surgical technique, its disadvantages are clearly known such that it is an important cause of mortality and morbidity, it definitely leads to a second surgery need, the patient is subjected to a double surgery risk and requirement of an experienced surgical team for adjusting the degree of band tightness. Besides, risks of pulmonary artery injury based on pulmonary band, occlusion of pulmonary arterial branches by band migration, secondary pulmonary valve insufficiency to pulmonary annulus dilatation, and subaortic stenosis development based on ventricular septal defect restriction in cases with aorta arising from hypoplasic ventricle are among potential complications specific to this operation. Despite all these drawbacks and disadvantages, the pulmonary artery banding operation particularly at neonatal and infancy period still continues to maintain its validity today and it is an important palliative surgery.

On the basis of the above-mentioned problems, a patent application having an application number of 2019/03805 and being titled as “Medical Device being Inserted Intravascularly and Providing Protection against Pulmonary Hypertension Risk in Treatment of Patients with Heart Defect” was made by me. Following implantation of the medical device, which is the subject of the said patent, to pulmonary artery; it may have a risk of thromboembolism albeit at a low rate as it may be in any medical device implanted to cardiovascular pathway. Therefore, an effective and accurate management is required for the potential risk.

To define thromboembolism briefly; it is a name given to a blood clot (embolism)—occurring intravenously—that breaks loose from the area where it first occurred, circulates by advancing along the vascular system and plugs another vessel (embolism).

Consequently, a requirement has occurred in order to realize an additional improvement and development in the medical device related to management of thromboembolism risk that is liable to occur after application of the product that is subject of the patent application numbered 2019/03805. A solution, which has been described. in literature for the first time, is provided against thromboembolism risk that may occur in any medical device implanted to cardiovascular pathway, albeit at a low rate.

### OBJECTIVE OF THE INVENTION

The main purpose of the present invention is to ensure management of thromboembolism risk that is liable to occur, albeit at a low rate, after implantation of the medical device (5) which is used for the purpose of palliative treatment and/or treatment without developing pulmonary hypertension in patients of all age groups who have human heart defects, particularly congenital heart defects (for example, heart defects with left right shunt or functional single ventricle), secondary increased pulmonary blood flow and pulmonary hypertension risk; and preparing the left ventricular for systemic circulation in case of development of right ventricular failure following atrial “switch” in patients who have transposition of large arteries with a left ventricular being subjected to involution—to the pulmonary artery. In addition, the adaptation capability of the inventive medical device for intracardiac hemodynamic changes is also enhanced.

In order to provide management of the above-mentioned potential thromboembolism risk, it has been enabled to direct blood flow in a controlled way by creating at least three orifices which are situated at the junction level of the proximal end close to the heart and the distal end of the medical device and on the cuffed distal end in a specific size and range.

### BRIEF DESCRIPTION OF THE DRAWINGS

FIG. 1 is a representative view of application of the inventive medical device which is inserted to main pulmonary artery intravascularly (into vessel) in order to keep the patient from pulmonary hypertension risk, in treatment of patients with heart defect.

FIG. 2 is a view of the inventive medical device ⁻while its application to the main pulmonary artery is in the starting position.

FIG. 3 is a view of the inventive medical device while it is applied to the main pulmonary artery.

FIG. 4 is a view of the orifices created on the device which enables to direct blood flow in a controlled way by providing management of a potential thromboembolism risk during application the inventive medical device.

### REFERENCE NUMBERS

1. Lung
2. Heart
3. Catheter systems
4. Main pulmonary artery
5. Medical device
   5.1 Proximal end
   5.2. Distal end
6. Orifice
7. Direction of blood flow

The drawings don't necessarily have to be scaled and details which are not necessary to understand the present invention may be omitted. Besides, members being at least substantially identical or having at least substantially identical. functions are indicated by the same number.

### DETAILED DESCRIPTION OF THE INVENTION

In this detailed description, the preferred configurations of the inventive medical device (5) are disclosed only for better understanding of the subject and such that they will not create any limiting effect.

The inventive medical device (5) will be inserted to the main pulmonary artery (4) by means of a catheter system (3), that is self-expandable and/or balloon expandable, by accessing to human body with a minimum interventional approach, intravenously in a percutaneous way. The above-mentioned medical device (5) comprises a part which provides an adherence area by reaching the main pulmonary artery (4) diameter and merging with the vessel on the proximal end (5.1) close to the heart (2) whereas it comprises a cuffed area with a smaller diameter that creates resistance to blood flow on the distal end (5.2). The said medical device (5) has a stent structure consisting of a two-piece fine metallic (nitinol, etc.) foldable and flexible cage (FIG. 1).

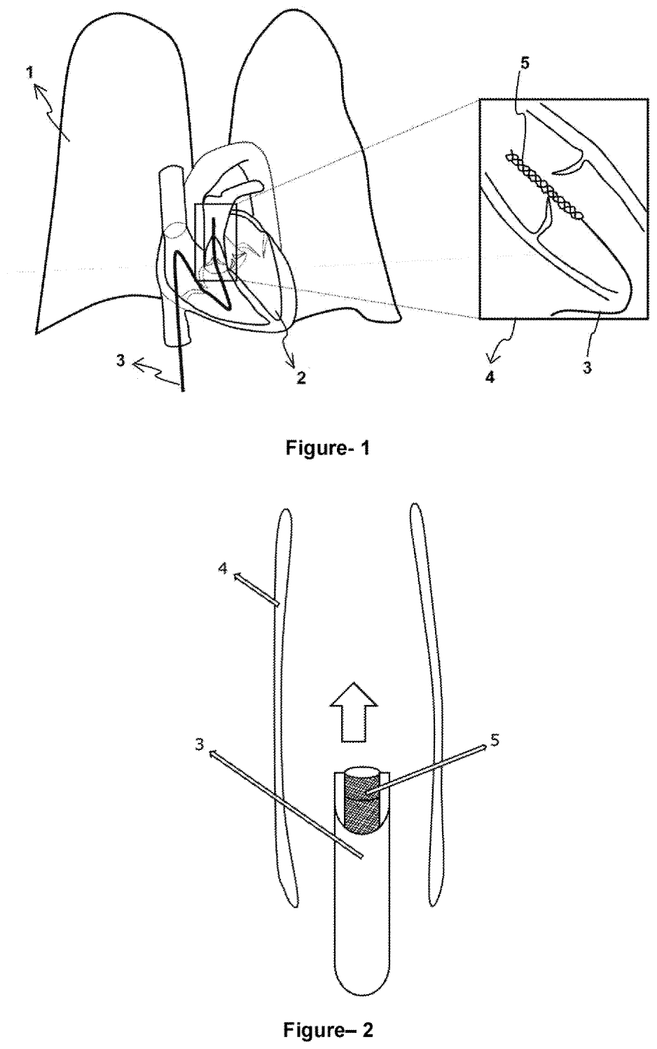

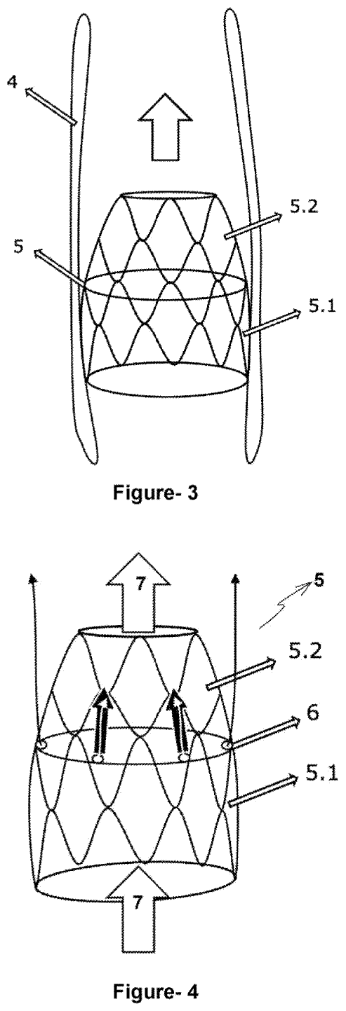

The inventive medical device (5) reaches into the vessel from the skin area under local anesthesia, with a minimally invasive approach to human body, and it is inserted to the main. pulmonary artery (4) upon being advanced along vascular structures and heart (2) by using wide wires, a catheter system (3) that is self-expandable and/or balloon expandable and can provide blood pressure monitoring. The catheter system (3) consisting of catheter and guide wires is used for transmitting the medical device (5) to the implantation site along artery or vein and for its implantation to the main pulmonary artery (4), at desired diameter (FIG. 2). The medical device (5), which is conveyed to the main pulmonary artery (4) in a folded way and with a diameter close to the catheter diameter, is carried safely along the cardiovascular pathway, it is conveyed to a second form by means of a catheter system (3) that is self-expandable and/or balloon expandable; the proximal end (5.1) diameter of the medical device (5) close to the heart is determined such that it will provide an adherence area whereas the cuffed distal end (5.2) diameter of the device is determined according to the blood pressure monitoring at the farthermost end of the catheter. A distal end (5.2) with a diameter providing a desired distal pulmonary blood pressure track is released and a “controlled” efficacy is ensured. The medical device (5) is inserted intravenously and fixed to the main pulmonary artery (4) by means of its flexible stent structure that consisting of wire-shaped elements that are linear and bendable on one another and expandable from each cell on request.

With the inventive medical device (5), blood flow directed to lungs (1) is limited at a “controlled” rate and by protecting both lung (1) site from high blood pressure, pulmonary hypertension risk is controlled and low pulmonary vascular resistance is reached. In addition, by balancing left-right pressure difference in heart (2), conditions that are considered to help spontaneous recovery of congenital heart defects are reached and thus, need for corrective treatment can be reduced. in the event that there is no need for limitation in lung (I) blood flow, the medical device (5) is released into vessel upon being dilated at a rate that can reach the main pulmonary artery (4) diameter with the catheter system that is self-expandable and/or balloon expandable, such that. it will not create any pressure difference in the main pulmonary artery (4) with a second minimally invasive intervention. Considering age groups and taking account of an achievable vessel diameter, dilatation degree and size of the stent is determined before implantation. Upon development of technological conditions, the device can be made of materials that are self-absorbable in body.

Following implantation of the inventive medical device (5) to pulmonary artery, it may have a risk of thromboembolism albeit at a low rate as it may be in any medical device implanted to cardiovascular pathway. In order to provide management of the said thromboembolism risk, at least three orifices (6) which are situated at the junction level of the proximal end (5.1) close to the heart and the distal end (5.2) of the medical device (5) and on the cuffed distal end (5.2) in a specific size and range according to size of the medical device (5) and which direct the blood flow have been created. It has been enabled to direct the blood flow in a controlled way by means of the orifices (6) situated on the said distal end (5.2). size and location of the orifices (6) are determined according to the medical device (5) area. The direction of blood flow is in the direction indicated in the FIG. 4 according to the location where the device is applied.

With this solution, risk of thromboembolism—that is expected in the area between the distal end (5.2) of the medical device (5) and the main pulmonary artery (4) and liable to develop depending on deceleration (stasis) of the circulation to a certain degree—is minimized. A continuous blood flow transition is allowed along the heart cycle in this area, at both systolic and diastolic phase, with the orifices (6). This management in blood flow actively provides solution against thromboembolism risk of the medicine after its intravenous insertion.

1. An additional feature of a medical device (5);
which is intended for palliative treatment and/or treatment without developing pulmonary hypertension in patients of all age groups who have human heart defects, particularly congenital heart defects, secondary increased pulmonary blood flow and pulmonary hypertension risk:
enables to prepare the left ventricular for systemic circulation in case of development of right ventricular failure following atrial “switch” in patients who have transposition of lame arteries with a left ventricular being subjected to involution;
reaches into the vessel from the skin area under local anesthesia, with a minimally invasive approach to human body; it is inserted to the main pulmonary artery (4) upon advancing the vascular structure along the heart by using a catheter system that is self-expandable and/or balloon expandable and can provide blood pressure monitoring;
characterized in that
it is an additional improvement in order to provide management of thromboembolism risk that may be associated with the medical device (5), after its implantation to the main pulmonary artery (4); it comprises at least three orifices (6) which are situated at the junction level of the proximal end (51) close to the heart and the distal end (5.2) and on the cuffed distal end (5.2).

2. A medical device (5) according to claim 1; characterized in that the size/area of the said orifice (6) vary by the size/capacity of the device.
